SEQUENCE LISTING (14 sequences)

SEQ ID NO: 1 - DNA, 38 bp, Artificial Sequence
  Primer
tattatgagctcttgaatgatcaaccaaacgccgatat

SEQ ID NO: 2 - DNA, 57 bp, Artificial Sequence
  Primer
tattatactagtttttttttttttttttttgtttgtttgtttgtttgtttactataa

SEQ ID NO: 3 - DNA, 537 bp, Saccharomyces cerevisiae
  CDS (1)..(537)
gcatccaacttgaacatttcgagaaagcttaccatatcaaccccatcatgctctttcgaaaattcaaatagcacatccattccttcgcccgcttcctcatctcaaagccacactccaatgagaaacatgagctcactctctgataacagcgttttcagccggaatatggaacaatcatcaccaatcactccaagtatgtaccaatttggtcagcagcagtcaaacagtatatgtggtagcaccgttagtgtgaatagtctggtgaatacaaataacaaacaaaggatctacgaacaaatcacgggtcctaacagcaataacgcaaccaatgattatattgatttgctaaacctaaatgagtctaacaaggaaaaccaaaatcccgcaacggcgcattacctcaatgggggcccacccaagacaagcttcattaaccatggaatgttcccctcgccaactgggaccataaatagcggtaaatctagcagtgcctcatctttaatttcttttggtatgggcaatacccaagtaatatag

SEQ ID NO: 4 - protein, 178 aa, Saccharomyces cerevisiae
ASNLNISRKLTISTPSCSFENSNSTSIPSPASSSQSHTPMRNMSSLSDNSVFSRNMEQSSPITPSMYQFGQQQSNSICGSTVSVNSLVNTNNKQRIYEQITGPNSNNATNDYIDLLNLNESNKENQNPATAHYLNGGPPKTSFINHGMFPSPTGTINSGKSSSASSLISFGMGNTQVI

SEQ ID NO: 5 - DNA, 36 bp, Artificial Sequence
  Primer
tattatgagctcggtgctgtgaccgtttccaatacg

SEQ ID NO: 6 - DNA, 44 bp, Artificial Sequence
  Primer
tattatactagttggtatttctgatgatgttcttgctctctttg

SEQ ID NO: 7 - DNA, 40 bp, Artificial Sequence
  Primer
tattatactagtatgagatttccttcaatttttactgcag

SEQ ID NO: 8 - DNA, 33 bp, Artificial Sequence
  Primer
tattatgtcgacttagtacattggttggccggg

SEQ ID NO: 9 - DNA, 70 bp, Artificial Sequence
  Primer
aagaagattacaaactatcaatttcatacacaatataaacgattaaaagacggatccccgggttaattaa

SEQ ID NO: 10 - DNA, 70 bp, Artificial Sequence
  Primer
tgggaacaaagtcgactttgttacatctacactgttgttatcagtcgggcgaattcgagctcgtttaaac

SEQ ID NO: 11 - DNA, 70 bp, Artificial Sequence
  Primer
ttactaccatcacctgcatcaaattccagtaaattcacatattggagaaacggatccccgggttaattaa

SEQ ID NO: 12 - DNA, 70 bp, Artificial Sequence
  Primer
atgaacgtgaaagaaatcgagagggtttagaagtagtttagggtcattttgaattcgagctcgtttaaac

SEQ ID NO: 13 - DNA, 33 bp, Artificial Sequence
  Primer
tattatgaattcatggtgagcaagggcgaggag

SEQ ID NO: 14 - DNA, 37 bp, Artificial Sequence
  Primer
tattatgtcgacttacttgtacagctcgtccatgccg

(19) United States
(12) Patent Application Publication
(10) Pub. No.: US 2011/0189657 A1
(43) Pub. Date: Aug. 4, 2011

(54) Device and a Method for the Detection and Amplification of a Signal

(75) Inventors: Kai Ostermann, Dresden (DE); Wolfgang Pompe, Hartha (DE); Gerhard Rodel, Karlsfeld (DE); Annett Gross, Dresden (DE)

(73) Assignee: TECHNISCHE UNIVERSITAET DRESDEN, Dresden (DE)

(21) Appl. No.: 12/666,303

(22) PCT Filed: Jun. 27, 2008
(86) PCT No.: PCT/EP2008/058280

§ 371 Date: Jun. 30, 2010

(30) Foreign Application Priority Data
Jun. 27, 2007 (DE) 10 2007 031 532.7

(51) Int. Cl. C12Q 1/68; C12M 1/34

(52) U.S. Cl. 435/6.1; 435/287.2

(57) ABSTRACT

The invention relates to a device and a method for the detection and amplification of a primary signal, utilizing an intracellular communication system, and the use thereof for the detection of substances such as phosphorus, sulfur, nitrogen, hormones, metabolic intermediates, fermentation products, and so forth. The device according to the invention for the detection and amplification of a primary signal contains cells of a first type for which a gene, which is responsible for the synthesis of a signal molecule, is under the control of a promoter which is regulated by the primary signal, and cells of a second type for which a specific gene is under the control of a promoter which is regulated by the separated signal molecule, in such a way that the secretion of the signal molecule is induced by a primary signal taken up by a cell of the first type, and the primary signal is amplified by the cells of the second type by the expression of the specific gene under the control of the signal molecule.

The invention concerns a device and a method for detection and amplification of a primary signal by utilizing an intercellular communication system and its use, for application in the detection of substances, for example, phosphorus, sulfur, nitrogen, hormones, metabolic intermediates, fermentation products etc.

Prior solutions for signal amplification have the disadvantage that substances can be detected only from a certain detection limit on.

U.S. Pat. No. 6,555,325 B1 discloses a system for detection of a functional interaction between a compound and the component of a cellular signal transduction cascade. The invention makes available a robust reproducible testing system for screening and identification of pharmaceutically active substances that modulate the activity of a cellular receptor or can interact with it. Especially, such substances are to be identified that interact with G-protein-coupled receptors that play an important role pharmaceutically. The yeast pheromone system is utilized here because the yeast pheromone receptor is also a G-protein-coupled receptor that with ectopically expressed G-protein-coupled receptor components can form heterologous receptors. For example, substances can be identified that act on non-yeast receptors. The detection of a functional interaction leads to secretion of a pheromone that in cells of the second mating type, in which a marker gene is under the control of a pheromone-responsive promoter excites the expression of this marker gene.

The object of the invention is to provide a device and a method for detection and amplification of a primary signal in order to lower the detection limit for the primary signal to be detected.

According to the invention, the object is solved by a device for detection and amplification of a primary signal that contains
a) cells of a first type in which a gene that is responsible for the synthesis of a signal molecule is under the control of a promoter that is regulated by the primary signal, and
b) cells of a second type in which a specific gene is under the control of a promoter that is regulated by the secreted signal molecule,

so that by a primary signal that is received by a cell of the first type the secretion of the signal molecule is induced and the primary signal is amplified by the signal molecule-controlled expression of the specific gene by the cells of the second type.

According to the embodiment of claim 2, additionally

c) cells of a third type are contained in which a gene that is responsible for the synthesis of a signal molecule is under the control of a promoter that is regulated by a signal molecule that is secreted by the cells of the first type,

so that
by a primary signal received by a cell of the first type the secretion of the signal molecule by the first cell is induced,
by the secretion of the signal molecule by the cells of the first type in the cells of the third type the secretion of the signal molecule is induced and a pre-amplification of the primary signal is effected, and
the primary signal is amplified by the signal molecule-controlled expression of the specific gene by the cells of the second type.

Cells have numerous communication systems by means of which they can exchange information with other cells. Often these communication systems are based on the secretion of a signal molecule by a cell into the surrounding medium. Other cells have suitable receptor systems for these signal molecules and can detect them and can react appropriately to them.

Such communication systems comprise, for example, quorum sensing of microorganisms, ammonia pulses in yeast cells, pheromone systems, for example in case of mating of yeast cells, secreted growth factors that within cell agglomerations are used for communication between the cells, virulence factors of microorganisms that specifically bond to eucaryotic receptors or immuno-modulating factors and factors affecting cell differentiations. The signal molecules that are secreted in these communications system are usable in the device according to the invention and the method according to the invention.

In case of quorum sensing, for example, microorganisms communicate by means of small hormone-like signal molecules. This process plays a decisive role in regulation of the cell density which enables bacteria populations to behave similar to a multi-cellular organism and therefore profit from advantages. The behaviorisms that are regulated by quorum sensing include inter alia the production of antibiotics, symbiosis, conjugation, virulence, formation of biofilms as well as bioluminescence of some Vibrio species. The signal molecules that are referred to as auto-inducers are produced by certain bacteria by means of certain genes and are released into the surrounding culturing medium. Bacteria have a matching receptor system for these ligands and, after a certain concentration of signal molecules in the medium is reached, can activate the transcription of certain genes. In this connection, there are species-specific auto-inducers with which the bacteria communicate within their own bacteria species. The so-called auto-inducer-2, on the other hand, enables the communication between different bacteria species (intraspecies communication).

In yeasts, periodically produced ammonia pulses serve as signals between colonies. In this connection, an ammonia pulse induces the ammonia production in neighboring colonies. This, in turn, has an effect on the spatial distribution of colonies because each ammonia pulse has the result that the growth zone between neighboring colonies is inhibited transiently. Accordingly, the colonies preferentially grow to the opposite side where no competing colonies are present.

In the device according to the invention the cells are in a liquid, preferably aqueous medium through which the exchange of signal molecules between the cells is realized. The cells of the device are either suspended in solution or immobilized on a carrier. The suspension is contained in a suitable container that ensures the measuring-technological detection of a signal emitted by the cells. The carrier is also designed such that signals emitted by the cells are detected by a detection system.

The invention can be used in all cells that have a receptor for a primary signal. In this connection, it is of no consequence whether this is an authentic or heterologous receptor. The same holds true for the optionally following signal cascade as well as the formation of signal molecules.

Advantageous embodiments of the invention are disclosed in claims 3 to 27.

According to the embodiment of claim 3, the cells of the first, second and/or third type are yeast cells, according to the embodiment of claim 4 the yeast cells are Saccharomyces cerevisiae or Schizosaccharomyces pombe cells.

The gene that'is responsible for the synthesis of the signal molecules is under the control of a promoter that is regulated by a primary signal. A promoter in genetics means a DNA sequence that controls the expression of a gene. Promoters in the meaning of the invention are preferably those areas of the genomic DNA that are responsible specifically for the regulation of the expression of a gene in that they react to specific intracellular or extracellular signals and, depending on these signals, will activate or repress the expression of the gene under their control.

In yeasts, these regulating DNA areas in general are on the 5′ end of the start codon of the respective gene and have an average length of 309 by (Mewes H. W. et al., (1997) Overview of the yeast genome. Nature 387, 7-65). Such regulating areas may however also be removed farther than 1,000 by from the coding sequence or may be positioned at the 3′ end of the coding sequence of the corresponding gene or even within the transcribing sequence of the corresponding gene. When such promoters are positioned at the 5′ end of the start codon of any gene, preferably a pheromone gene, they regulate the activity of this gene as a function of the above-mentioned specific primary signals.

The primary signals that are detected by the yeast are either signals for which the yeast cell has its own receptor systems. This may be, for example, signals that indicate the deficiency of certain nutrients essential for the yeast cell, for example, nitrogen, sulfur, phosphorus, iron or copper, carbon sources, essential amino acids, oxygen, or other signals such as temperature, DNA damage, ER stress, or oxidative stress.

The cell of the first type according to the embodiment of claim 5 may however be modified gene-technologically such that it expresses a receptor systems that is not contained in the cell naturally. The primary signals that are received by these receptor systems are of chemical nature, for example, ions, inorganic and organic compounds and biomolecules such as proteins, peptides, lipids, sugars or nucleic acids or also of physical nature, for example, electromagnetic radiation, pressure, temperature or conductivity.

For example, the expression of such receptor systems in yeast cells is known in the prior art. For example, in yeasts sensor systems can be expressed for human hormones such as androgens (Bovee, T. F. H et al. (2008) A new highly androgen specific yeast biosensor, enabling optimisation of (Q)SAR model approaches. The Journal of steroid biochemistry and molecular biology 108:121-131), for heavy metals such as cadmium (Park, J.-N. et al. (2007) Identification of the cadmium-inducible Hansenula polymorpha SEQ1 gene promoter by transcriptome analysis and its application to whole-cell heavy-metal detection Systems. Applied and environmental microbiology 73:5990-6000), for volatile olfactory substances (Marrakchi, M. et al. (2007) A new concept of olfactory biosensor based on interdigitated microlelectrodes and immobilized yeasts expressing the human receptor OR17-40. European Biophysical Journal 36:1015-1018) and even explosives (Radhika, V. et al. (2007) Chemical sensing of DNT by engineered olfactory yeast strain. Nature Chemical Biology 3:325-330).

Since the cells are in a liquid medium, the primary signals to be amplified must also be either in this liquid medium or, as in case of some physical parameters as, for example, temperature, must be transmitted through this medium to the cells of the device according to the invention.

When the cells of the first type detect by means of these endogenic or recombinant receptors the incoming primary signals, directly or indirectly by means of intermediately positioned signal cascades, the transcription of the signal-specific promoter is induced so that the cells of the first type as a response to the incoming primary signal secret the signal molecule into the environment.

The cells of the second type have on their surface receptors for the signal molecule that has been secreted by the cells of the first type as well as for the signal cascade that is required for the intracellular transmission of the signal and is optionally modified by means of molecular-biological techniques. The cell of the second type according to the embodiment of claim 6 can also be gene-technologically modified such that it expresses a receptor for a signal molecule that the cell does not possess naturally.

The cells of the second type are according to the invention genetically modified such that a specific gene that, for example, codes for a marker protein, for example, GFP (green fluorescent protein), is under the control of a promoter that is regulated by the signal molecule that is secreted by the cells of the first type.

When the signal molecule secreted by the cells of the first type as a response to the specific primary signal reaches the surrounding cells of the second type, in these cells of the second type the expression of the specific gene and the production of the marker protein or target protein is strongly induced by the signal molecule-regulated promoter.

In another embodiment of the invention, the specific gene itself is again responsible for the synthesis of a signal molecule that is secreted by the cell of the second type. In this way, it is advantageously possible to connect several devices of the present invention in the form of a cascade of amplifiers. The signal molecule that is secreted by the cells of the second type is detected by a second device according to the invention as a primary signal and is therefore further amplified. This cascade can be extended at will by the use of different signal molecules. At the end of the cascade, there is typically a cell that has a specific gene that is signal molecule-regulated that codes for a marker protein, for example, GFP (green fluorescent protein) so that the primary signal, amplified several times, can then be detected by a suitable detection system.

Yeast cells can be present in the diploid state as well as in the haploid state. Two haploid yeast cells can be combined in a process that is referred to as mating to a single diploid yeast cell. In case of haploid yeast cells a differentiation is made between two so-called mating types. Only yeast cells with different mating types can mate with one another. For example, these are in case of bakers yeast Saccharomyces cerevisiae the mating types α and a, in case of the fission yeast Schizosaccharomyces pombe the mating types plus and minus.

Haploid yeast cells communicate by so-called pheromones. These are short peptides formed by the respective cells in order to communicate to their environment their own mating type. Saccharomyces cerevisiae yeast cells of the mating type a secret, for example, the pheromone α-factor and Saccharomyces cerevisiae yeast cells of the mating type a secrete the pheromone a-factor.

According to the invention, cells of the first type are genetically modified such that a gene that is responsible for the synthesis of the signal molecule is subjected to the control of a promoter that is regulated by the primary signal to be detected. In this connection, the gene itself codes for the signal molecule or a protein that effects the synthesis of the signal molecule in the cell and/or its secretion. According to the embodiment of claim 7, this signal molecule is preferably a pheromone.

Pheromones in the meaning of the present invention are, aside from the naturally occurring pheromones in yeast cells, also homologous or modified peptides or peptide analogues, or other organic compounds that are capable of binding to and activating pheromone receptors of yeast cells.

The gene that codes for the pheromone can either be a natural gene that is contained in the genome of an organism or a synthetic gene sequence whose expression causes the production of a pheromone or a peptide that is homologous to a pheromone that is capable of activating the pheromone receptors of yeast cells.

In a preferred embodiment, the invention thus is comprised of a device for detection and amplification of a primary signal with

a) haploid cells of a first type in which a gene that codes for a pheromone is under the control of a promoter that is regulated by a primary signal, and

b) haploid cells of a second type in which a specific gene is under the control of a promoter that is regulated by the pheromone to be secreted,

so that by a primary signal that is taken up by a cell of the first type the secretion of the pheromone is induced and the primary signal is amplified by the pheromone-controlled expression of the specific gene by the cells of the second type.

The cells of the first type, according to the embodiment of claim 8, are Saccharomyces cerevisiae cells of the a mating type or Saccharomyces cerevisiae cells of the mating type a or diploid Saccharomyces cerevisiae cells.

According to the embodiment of claim 9, the cells of the second type are Saccharomyces cerevisiae cells of the a mating type or Saccharomyces cerevisiae cells of the mating type a.

The cells of the third type, according to the embodiment of claim 10, are Saccharomyces cerevisiae cells of the a mating type or Saccharomyces cerevisiae cells of the mating type a.

The yeast cells have on their surfaces receptors for the pheromones of the respective opposite mating type. For example, Saccharomyces cerevisiae cells of the mating type a are capable of recognizing Saccharomyces cerevisiae cells of the mating type a in their environment and vice versa.

The gene that is responsible in the cells of the first and/or third type for the synthesis of the signal molecule is, according to the embodiment of claim 11, the MFα1 or MFα2 gene of Saccharomyces cerevisiae that codes for the pheromone α-factor, respectively, or the MFA1 gene or the MFA2 gene of Saccharomyces cerevisiae that codes for the pheromone a-factor.

The promoter that is regulated by the pheromone used as a signal molecule is, according to the embodiment of claim 12, the FIG1 promoter of Saccharomyces cerevisiae.

The transcription of the FIG1 gene is increased by up to 97 times after incubation of haploid yeast cells with pheromones of the respective opposite mating type (Roberts, C. J., et al. (2000) Signaling and circuitry of multiple MAPK pathways revealed by a matrix of global gene expression profiles. Science 287:873-880). Originally, it was identified in a yeast “two-hybrid screen” for identification of pheromone-regulated genes (Erdman, S., et al. (1998) Pheromone-regulated genes required for yeast mating differentiation. Journal of Cell Biology 140: 461-483). The name FIG1 means “factor induced gene 1”. It is an integral membrane protein that, directly or indirectly, participates in the Ca2⁺ uptake in the cell (Muller, E. M. et al. (2003) Fig1p facilitates Ca2+ influx and cell fusion during mating of Saccharomyces cerevisiae Journal of Biological Chemistry 278: 38461-38469). After addition of α-factor to a-cells, in the latter a significant increase of the protein after 60 minutes is detectable (Roberts et al., 2000). At the transcription level, an increase of the mRNA concentration by more than 97 times is observed already 20 minutes after addition of the respectively used pheromone to the yeast cells(Roberts et al., 2000). The promoter of the FIG1 gene is therefore advantageously highly regulatable by the pheromone of the opposite mating type, respectively. Accordingly, the effects of pheromones can be detected quickly and sensitively.

An DNA segment is preferred as a promoter that comprises up to 1,000 by at the 5′ end of the start codon of the FIG1 gene or a section of this DNA segment that is capable of, in the presence of the pheromone, activating or repressing the specific gene that is under the control of this sequence.

Especially preferred is the use of the DNA segment that is obtained by. PCR amplification of Saccharomyces genome by use of the primers Fig1 p-for (SEQ NO. 1) and Fig1-rev (SEQ NO. 2) (see Table 1).

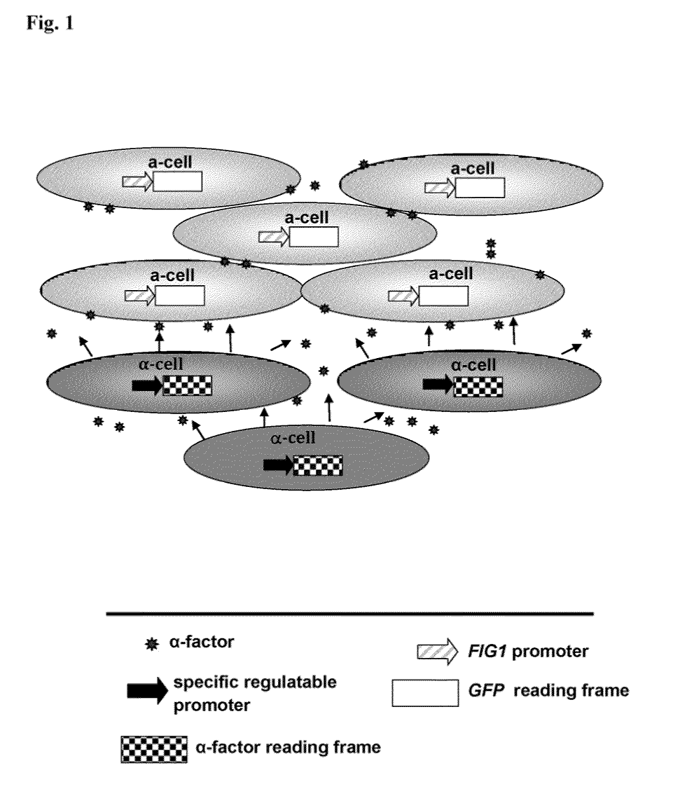

Table 1 Primer for amplification of the FIG1 promoter. The letters in bold print delimit the utilized promoter segment of the FIG1 gene. The recognition sequences of the restriction endonucleases Sad and Spe1 used for cloning are shown in italics: The first six bases serve for protecting the primer.

| SEQ | | |
|---|---|---|
| NO. | Primer | Sequence (5′ → 3′) |
| 1 | FIG. 1- | TAT TAT GAG CTC TTG AAT GAT CAA CCA |
| | for | AAC GCC GAT AT |
| 2 | FIG. 1- | TAT TAT ACT AGT TTT TTT TTT TTT TTT |
| | rev | TTT GTT TGT TTG TTT GTT TGT TTA CTA |
| | | TAA |

Promoters in the meaning of the invention are also DNA segments that have in comparison to the corresponding yeast promoters a homology of more than 50%, preferably more than 80%. These sections may originate, for example, from homologous genomic areas of other organisms, preferably other yeast strains. They can however also be synthetically produced DNA sequences whose sequence has a homology of more than 50%, preferably more than 80%, identity with the corresponding Saccharomyces cerevisiae promoter. Promoters can also be synthetic DNA sequences that are combined of a partial section of one of the aforementioned yeast promoters as well as a known basal promoter of Saccharomyces cerevisiae.

The basal promoter provides the DNA sequences required for connecting the transcription machinery while the partial sequences of the yeast promoters react specifically to regulating signals. Such a basal promoter is preferably the basal promoter of cytochrome c gene of Saccharomyces cerevisiae that comprises 300 by at the 5′ end of the start codon of cytochrome c gene (Chen, J. et al. (1994) Binding of TFIID to the yeast CYCI TATA boxes in yeast occurs independently of upstream activating sequences. Proc. Natl. Acad. Sei. USA 91:11909-11913).

Promoters of synthetic DNA sequences may contain also multiple segments of an identical DNA sequence. This multiplication of a regulatory DNA segment enables advantageously an increase of the sensitivity of the promoter relative to the signals to be detected.

In the process of mating of yeast cells the transcription factor Ste12p induces the expression of the pheromone-responsive genes by binding of Ste12p to so-called “pheromone responsive elements” (PREs) in the promoter region of inducible genes (Dolan et al., (1989). The yeast STE12 protein binds to the DNA sequence mediating pheromone induction. Proc. Natl. Acad. Sci. USA 86: 5703-5707.). Hagen et al. have demonstrated that tandem-like arranged PREs are sufficient in order to activate the pheromone-responsive expression of haploid-specific genes in both mating types (Hagen et al. (1991). Pheromone response elements are necessary and sufficient for basal and pheromone-induced transcription of the FUS1 gene of Saccharomyces cerevisiae. Mol. Cell. Biol. 11:2952-2961). PREs are elements of 7 by length with the consensus sequence TGAAACA (Kronstad et al., (1987). A yeast Operator overlaps an upstream activation site. Cell 50: 369-377.).

In the FIG1 promoter three putative binding locations for Ste12p have been identified (Harbison et al., (2004). Transcriptional regulatory code of a eukaryotic genome. Nature 431: 99-104.).

The response time of the FIG1 promoter can be shortened by a higher number of PREs. For example, in addition or in place of the authentic activator section of the gene, a fragment of a length of 139 by with the PREs of the regulatory region of FUS1 or a simple synthetic cluster of PREs can be used (Hagen et al.; 1991). Advantageously, with a reporter construct of the modified FIG1 promoter and an EGFP marker a higher expression of the marker genes and improved response capability to reduced pheromone concentrations is obtained. Also, a temporally faster response of the system is achieved.

According to the embodiment of claim 13, in the cells of the second type the transcription activator Ste12p is overexpressed. The overexpression of STE12 causes an increased expression of pheromone-responsive genes, mediated by PREs (Dolan and Fields, (1990). Overproduction of the yeast STE 12 protein leads to constitutive transcriptional induction. Genes Dev. 4: 492-502). Advantageously, by overexpression of the transcription activator Ste12p also the expression level of the specific gene under the control of the pheromone-dependent promoter is increased.

In order to bind to individual PREs, Ste12p requires partially further transcription activators such as the factor Mcm1p (Hwang-Shum et al, Jr (1991). Relative contributions of MCM1 and STE12 to transcriptional activation of a- and α-specific genes from Saccharomyces cerevisiae. Mol Gen Genet 227: 197-204.). According to the embodiment of claim 14, in the cells of the second type Mcm1p is therefore overexpressed. The heterogeneous expression of this factor contributes also to increase of the expression of the specific gene that is under the control of the pheromone-dependent promoter.

The specific gene, according to the embodiment of claim 15, mediates the formation of the signal molecule that is different from the signal molecule that is secreted by the cells of the first type. In this way, advantageously several devices according to the invention can be switched in series as a cascade.

The specific gene codes, according to the embodiment of claim 16, for a marker protein, preferably a fluorescent protein such as GFP (green fluorescent protein) or an enzyme such as β-galactosidase.

By means of a detection system for the marker protein, for example, GFP, the signal-induced formation of the marker protein can be detected sensorically (detection).

Marker proteins in the meaning of the invention are proteins whose presence or activity leads to a physically measurable change. This physically measurable change can be detected by a suitable detection system in a simple and quick way. Preferably, such marker proteins are used that can detect without impairing the integrity or vitality of the cells, for example, by enzymes that catalyze in the presence of a substrate a color reaction, such as β-galactosidase or phytase. Further preferred as a marker protein are luciferases that, in the presence of a suitable substrate, emit light. Especially preferred as marker proteins are proteins that by excitation with light fluoresce at a certain wavelength.

Furthermore, the invention comprises as marker proteins proteases that decompose fluorescent proteins. When the yeast cell expresses at the same time constitutively a fluorescent proteins, in response to the primary signal the decrease of the fluorescence of the cells of the second type is measurable. Preferably used are proteins that, aside from the fluorescent protein, do not attack further targets in the cell in order not to impair the vitality of the cell. Especially preferred is the use of TEV protease. The appropriate fluorescent proteins must optionally be modified by means of recombinant DNA techniques such that they contain the recognition sequence for the corresponding protease and are therefore decomposable.

According to the embodiment of claim 17, a marker protein is a fluorescent protein wherein the expression of the corresponding marker protein after secretion of the signal molecule by the cells of the first type varies which causes increase or decrease of the fluorescence of the respective yeast cell. Preferably used are the fluorescent proteins GFP, YFP, CFP, BFP, RFP, DsRed, PhiYFP, JRed, emGFP (“Emerald Green”), Azami-Green, Zs-Green or AmCyan 1. Preferably used are proteins that have been modified such that they fluoresce especially strongly, such as eGFP, eYFP, TagCFP, TagGFP, TagYFP, TagRFP and TagFP365. Moreover, preferably used are those fluorescent proteins whose amino acid sequence has been modified such that they begin to fluoresce as quickly as possible after their formation. Preferably used are also TurboGFP, TurboYFP, TurboRFP, TurboFP602, TurboFP635, and dsRed-Express.

According to the embodiment of claim 18, the modified gene codes for a fluorescent protein as a marker protein that fluoresces green (for example, GFP), yellow (for example, YFP), blue (for example, BFP), cyan (for example, CFP) or red (for example, dsRed).

For monitoring as early as possible it may be advantageous to destabilize the corresponding marker proteins by adding sequences that lead to increased “turnover” of the proteins. According to the embodiment of claim 19, the marker protein is a fluorescent proteins with limited half-life. In this way, a quick response time upon decrease of the transcription is ensured.

Such a limited half-life can be achieved, for example, by modification of the N-terminal amino acid or the introduction of a signal sequence into the amino acid sequence of the protein that is coded by the marker gene so that the stability of the protein is lowered and its half-life is shortened. For the destabilization of the protein that is coded by the marker gene a so-called PEST domain is used preferably that leads to a fast decomposition of the protein by the ubiquitin system of the cell. Such PEST domains are known from many proteins. The PEST domain of the G1 cyclin Cln2p of Saccharomyces cerevisiae is preferably used. For this purpose, onto the 3′ end of the coding sequence of the marker gene the coding sequence (SEQ NO 3) of 178 carboxyl-terminal amino acids of Cln2p (SEQ NO. 4) and a stop codon are added.

```
CIn2p Pest Sequence (SEQ NOS: 3/4):
GCATCCAACTTGAACATTTCGAGAAAGCTTACCATATCAACCCCATCATGCTCTTTCGAAAATTCAAATAGCACA
  A S N L N I S R K L T I S T P S C S F E N S N S T
TCCATTCCTTCGCCCGCTTCCTCATCTCAAAGCCACACTCCAATGAGAAACATGAGCTCACTCTCTGATAACAGC
  S I P S P A S S S Q S H T P M R N M S S L S D N S
GTTTTCAGCCGGAATATGGAACAATCATCACCAATCACTCCAAGTATGTACCAATTTGGTCAGCAGCAGTCAAAC
  V F S R N M E Q S S P I T P S M Y Q F G Q Q Q S N
AGTATATGTGGTAGCACCGTTAGTGTGAATAGTCTGGTGAATACAAATAACAAACAAAGGATCTACGAACAAATC
  S I C G S T V S V N S L V N T N N K Q R I Y E Q I
ACGGGTCCTAACAGCAATAACGCAACCAATGATTATATTGATTTGCTAAACCTAAATGAGTCTAACAAGGAAAAC
  T G P N S N N A T N D Y I D L L N L N E S N K E N
CAAAATCCCGCAACGGCGCATTACCTCAATGGGGGCCCACCCAAGACAAGCTTCATTAACCATGGAATGTTCCCC
  Q N P A T A H Y L N G G P P K T S F I N H G M F P
TCGCCAACTGGGACCATAAATAGCGGTAAATCTAGCAGTGCCTCATCTTTAATTTCTTTTGGTATGGGCAATACC
  S P T C T I N S G K S S S A S S L I S F G M G N T
CAAGTAATATAG
  Q V I *
```

The gene that is under the control of a promoter specific for a signal molecule that codes for a marker protein or is responsible for the synthesis of a signaling molecule is introduced into a cell, preferably a yeast cell. In this connection, it may be present on an extrachromosomal DNA molecule. Preferably used for this purpose is a yeast expression vector that upon division of the yeast cell replicates stably. Especially preferred is a so-called “high copy number vector that exists in the cell in a large number of copies. Alternatively, also vectors are used which in minimal copy numbers or as an individual vector are present in yeasts, for example, ARS-CEN vectors or yeast artificial chromosomes.

In another embodiment the gene is integrated together with the pheromone-specific promoter into the chromosomal DNA of the yeast cell. In this way, advantageously it is ensured that all progeny of the yeast cell also contain the marker gene under the control of the specific promoter.

The detection and amplification device according to the invention has the advantage that a primary signal that is received by a cell of the first type is amplified many times by the thus induced secretion of the signal molecule and its effect on the surrounding responsive cells of the second type.

In this connection, with a targeted influencing of the numerical ratios of the different cell types the amplification effect can be further enhanced. For a random distribution of the cells as cell mixture, the cells of the first type relative to the cells of the second type are present in the ratio of 1 to 20, preferably 1 to 10, particularly preferred 1 to 5. In composite structures with an expediently structured distribution of the individual cell types in granular or layered phases the optimal concentration ratio is additionally a function of the selected spatial arrangement of the cells relative to one another.

Moreover, amplification and sensor systems that are based on living cells have the great advantage of natural regeneration of the employed components. This is beneficial in particular in methods of “on-line monitoring” but also “near-line monitoring” of processes.

The biological detection and amplification device according to the invention can be utilized, for example, in order to:
detect early on the limitation of substances such as phosphate, sulfur, nitrogen or a carbon source, for example, glucose, in a culturing medium:
more effectively detect loading of cells by stress factors, for example, radiation or chemical agents;
detect the occurrence of target molecules, for example, certain metabolic intermediates at an early stage;
design more effectively bioassays for the detection of substances in aqueous solutions.

In the yeast cells of the first and/or second and/or third mating type according to the embodiment of claim 23 the authentic regulation of the expression of pheromones is turned off. For example, the natural genes MFα1 and MFα2 in α-cells of Saccharomyces cerevisiae cells according to the embodiment of claim 21 are deleted. According to the embodiment of claim 22, the natural genes MFA1 and MFA2 are deleted in Saccharomyces cerevisiae cells of the mating type a. In this way, it is advantageously ensured that the α-factor or the a-factor are formed and secreted exclusively when the primary signal to be detected is present. Secondary effects on the cells of the second type are prevented in this way.

According to the embodiment of claim 23 on the yeast cells of the first and/or second and/or third mating type the protein Fig1p is inactivated. High local concentrations of pheromones trigger cell death in yeast cells. By inactivation of Fig1 p this effect is prevented (Zhang, N. -N., et al. (2006) Multiple signaling pathways regulate yeast cell death during response to mating pheromones. Mol. Biol. Cell 17: 3409-3422). In this way, it is prevented advantageously that the yeast cells as a result of a high concentration of pheromone caused by a strong primary signal would die off and thus no longer be available for the method (Zhang et al., 2006).

The promoter that can be controlled by the primary signal, according to the embodiment of claim 24, is a nitrogen-, phosphate- or sulfur-specific regulated promoter. The primary signal to be amplified is nitrogen, phosphate, or sulfur deficiency.

Preferably, as a promoter a DNA segment is employed that comprises up to 1,000 by at the 5′ end of the start codon of the gene controlled by it, or a partial section of this DNA segment that, upon deficiency of nitrogen, phosphorus or sulfur, is capable of activating or repressing the marker gene under the control of this sequence.

The nitrogen-specific, phosphate-specific or sulfur-specific regulated promoter is according to the embodiment of claim 25 selected from the promoters of the genes YIR028W, YJR152W, YAR071W, YHR136C, YFLO55W and YLL057C of Saccharomyces cerevisiae.

Promoters of genes whose transcription as a response to a corresponding limitation is greatly increased, are advantageously in case of
nitrogen imitation: YIR028W and YJR152W;
phosphate limitation; YAR071 Wand YHR136C;
sulfur limitation: YFL055W and YLL057C.

According to the embodiment of claim 26, the cells are disposed in a porous organic or inorganic gel, according to the embodiment of claim 27 in a porous and optically transparent silicon dioxide xerogel.

Advantageously, the cells are immobilized in xerogels. Xerogels are gels that have lost their liquid, for example, by evaporation or applying vacuum. Gels are shape-stable, easily deformable disperse systems of at least two components that are comprised usually of a solid material with elongate or greatly branched particles (for example, silicic acid, gelatin, collagens, polysaccharides, pectins, special polymers, for example, polyacrylates, and other gelling agents that are frequently referred to as thickening agents) and a liquid (usually water) as a dispersion medium. In this connection, the solid substance in the dispersion medium produces a three-dimensional network. When xerogels are formed, the three-dimensional arrangement of the network changes.

The use according to the invention of inorganic or biologically inert organic xerogels for embedding the cells enables advantageously the survival of the cells while providing simultaneously stability of the produced structures because they are toxicologically and biologically inert and in general are not decomposed by the cells. They enable moreover advantageously the incorporation of nutrients and moisturizing agents that ensure survival of the cells.

Advantageously, the cells are immobilized in a porous and optically transparent inorganic or biologically inert organic xerogel. Preferably, the xerogel is an inorganic xerogel of silicon dioxide, alkylated silicon dioxide, titanium dioxide, aluminum oxide or their mixtures. It is preferred that the inorganic xerogel is produced preferably by a sol-gel process.

For this purpose, first silica sols or other inorganic nanosols are produced either by acid-catalyzed or alkali-catalyzed hydrolysis of the corresponding silicon alkoxide or metal alkoxide in water or in a water-soluble organic solvent (such as ethanol). Preferably, hydrolysis is carried out in water in order to prevent toxic effects of the solvent on the cells to be embedded. When producing nanosols by alkoxide hydrolysis, during the course of the reaction alcohols are produced that are subsequently evaporated from the obtained nanosol by passing through an inert gas flow and replaced by water.

By using mixtures of different alkoxides the matrix properties can be affected in a targeted fashion. The sol-gel matrix enables advantageously the chemical modification by co-hydrolysis and co-condensation by utilizing different metal oxides of metals such as Al, Ti, Zr for producing mixed oxides or of alkoxy silanes with organic residues on the Si atom for producing organically modified silicon oxide gels.

The cells to be embedded are mixed with the resulting nanosol. The process of gel formation is initiated preferably by increasing the temperature, neutralizing the pH value, concentration, or addition of catalysts, for example, fluorides. However, in this connection, the temperature should not be increased to temperatures of >42° C. in order not to damage the cells to be embedded. When converting into a gel, the nanosols reduce their surface area/volume ratio by aggregation and three-dimensional cross-linking. During this conversion of the nanosol into a so-called lyogel, the cells are immobilized in the resulting inorganic network. The immobilization of cells capable of survival is advantageously controlled by the ratio of cells to oxide and by addition of pore-forming agents.

The proportion of cells in the total quantity of the generated xerogel including the embedded cells, depending on the application, can be from 0.1 to 50% by weight. Preferred is a proportion of 2 to 25% by weight.

By drying, solvent that is still contained in the lyogel is removed. In this way, the gel is converted to the xerogel. The resulting xerogel has a high porosity that enables fast material exchange with the surrounding medium. The drying process causes a great shrinkage of the gel that leads to stress for the embedded cells. Preferably, the drying step is therefore performed very gently and slowly at temperatures of less than 40° C.

With decreasing water content of the matrix, the physiological activity and the survival rate of the embedded cells will drop. A water content that is too high leads however to low mechanical stability and reduces the durability of the structure.

The use of yeast cells is particularly advantageous because yeast cells have a high resistance with respect to dryness and even at very minimal water contents do not lose their survival capability. In this way, it is possible to produce very dry xerogels.

The invention comprises also the use of different additives such as soluble organic salts, i.e., metal salts of organic carboxylic or sulfonic acids or open-chain or cyclic ammonia salts and quaternary salts of N-heterocycles as well as low-molecular polyanions or polycations, or water-soluble organic compounds such as poly carboxylic acids, urea derivatives, carbohydrates, polyols, such as glycerin, polyethylene glycol and polyvinyl alcohol, or gelatin, that act as plasticizers, moisturizing agents and pore forming agents, inhibit cell lysis, and increase significantly the survival capability of the embedded cells.

According to the embodiment of claim 28, the silicon dioxide xerogel with the cells is disposed on a substrate with increased mechanical stability. The inorganic xerogel for this purpose is applied with the cells onto a substrate. In connection with the signal detector, preferably a photodetector, a functional element is thus provided wherein the fluorescent light produced as a function of the bioavailable analytes is converted by the photodetector into an electrical signal. As a further development, the substrate, according to the embodiment of claim 29, is advantageously an optical fiber, glass beads, a planar glass support or other shaped bodies of glass such as hollow spheres, rods, tubes or ceramic granules.

In this connection, the cells are positionally fixed in a porous and optically transparent inorganic xerogel, for example, a silicon dioxide xerogel. The silicon dioxide xerogel to which the microorganisms have been added is deposited as a layer onto glass beads, an optical fiber, planar glass supports or other shaped bodies such as hollow spheres, rods, tubes or ceramic granules by means of a known sol-gel process in that the nanosol/cell mixture is applied onto the substrate is applied onto the substrate to be coated or the substrate is immersed into the nanosol-cell mixture and the nanosol is subsequently transformed by drying and the thus resulting concentration of the nanosol into a xerogel. The thus obtained mechanical stability of these structures enables the introduction of the device according to the invention into a measuring system that can be connected immediately with the reaction space (fermenter) to be examined in the context of near-line diagnostics.

According to the embodiment of claim 30, the cells are a component of an envelope structure that surrounds at least partially a cavity. This means that individual or several cells are encapsulated in this cavity that has a porous envelope. The microporosity enables advantageously a material exchange with the environment. In a further development, the envelope structure according to the embodiment of claim 31 is comprised of a base member with an inner layer of a biological hydrogel and an outer layer of the porous and optically transparent silicon dioxide xerogel wherein the layers are applied at least partially.

The cells in this connection are embedded in the envelope structure (duplex embedding). The inner envelope is comprised of a biological hydrogel, for example, alginate, and the outer envelope is a porous xerogel layer, preferably an inorganic xerogel layer, especially preferred a silicon dioxide xerogel layer. The biological hydrogel stabilizes advantageously the cells in the subsequent process of coating with the silicon dioxide sol and increases thus the survival probability of the cells. This duplex embedding can advantageously be realized by a sequential coating by utilizing a nano plotter. The mechanical stability of such structures enables the application of the device according to the invention into the reaction space to be examiner (fermenter) in the context of near line diagnostics.

The cells according to the embodiment of claim 31 are embedded in a structure with a hierarchical pore structure wherein, in addition to the nanoporosity typical for inorganic gels, the structure is additionally penetrated by mesopores connected to one another whose diameter typically varies between 100 nm to 100 μm and that enable the material echange between the environment and the embedded cells as well as their reaction products such as the enzymes.

In this way, advantageously the cells of the first type and the cells of the second type as well as optionally cells of the third type can be applied in different layers on the substrate. Because of the nanoporosity the specific primary signals can reach the cells of the first type located in the outer layer of the sensor and also the pheromones secreted by these cells can reach the cells of the second and/or third type that are located in the layer underneath that has direct contact with the signal detector.

With respect to the spatial arrangement of the three yeast cell types in layers, there are three basic options of distribution of the cells in a cell mixture, in a composite structure that is comprised of different structural building blocks with different cell distributions, or as a graduated layer system with a depth-dependent continuous change of the concentration distribution of the three cell types.
1. Cell Mixture
Cells of the type 1 and type 2 as well as optionally cells of the type 3 are immobilized in a predetermined quantity ratio for adjusting the desired amplification level into a solid matrix or, alternatively, introduced into an aqueous solution in a statistically random distribution.
Advantages of these cell mixtures are the short transport paths for the exchange of the signal molecules between the individual cells as well as the statistical homogeneity of the sensor material. A disadvantage may result from the limited accessibility of the type 1 cells (arranged in the depth of the sensor material) for the external primary signal.
With the concentration ratios realized in the cell mixture of the three cell types the amplification level can be adjusted in the desired way.
   2. Composite Structure
The composite structure can be configured as needed of granules or individual layers (in regard to the individual layers see also FIG. 3).
In the granules one or two cell types are immobilized, respectively. With a suitable arrangement of the granules relative to one another as well as a suitably selected mixing ratio of the cells in the granules as well as the granules relative to one another, the amplification level can be adjusted: advantageously, the proportion of granules with the cell type 1 is increased in the external area of the composite structure for an effective reception of the external primary signal. The granules with the cell types 1 and 3 serve for pre-amplification of the external primary signal. The granules with the cell types 1 and 2 or 3 and 2 serve for final amplification and conversion into the physical, chemical or biochemical signal that is to be read out.
An analog approach is provided for the arrangement of the three cell types in layer systems. In this connection, in individual layers one or two cell types are embedded. Layers with the cell type 1 are advantageously arranged in the external area of the composite structure in order to ensure effective reception of the external primary signal. Individual layers with the cell types 1 and 3 serve for pre-amplification of the external primary signal. Individual layers with the cell types 1 and 2 or 3 and 2 serve for final amplification and conversion into the physical, chemical or biochemical signal to be read out. The layers can be applied in a flat geometry on a suitable support. However, layer-shaped concentric structural arrangements as well as the coating of randomly curved supports are also encompassed by the invention.
   3. Graduated Layers
Graduated layers represent a transition from the discrete cell distribution in the layer system to the cell mixtures in that by a suitable coating strategy a quasi continuous change of the concentration distribution of the three cell types from the exterior (preferably type 1 and type 3 cells) to the area of the reading structure (preferably cells of the type 2 and 3) is realized. A graduated layer combines the advantage of an effective reception of the external primary signals with transport paths as short as possible for the biological intracellular signal molecules within the device according to the invention.

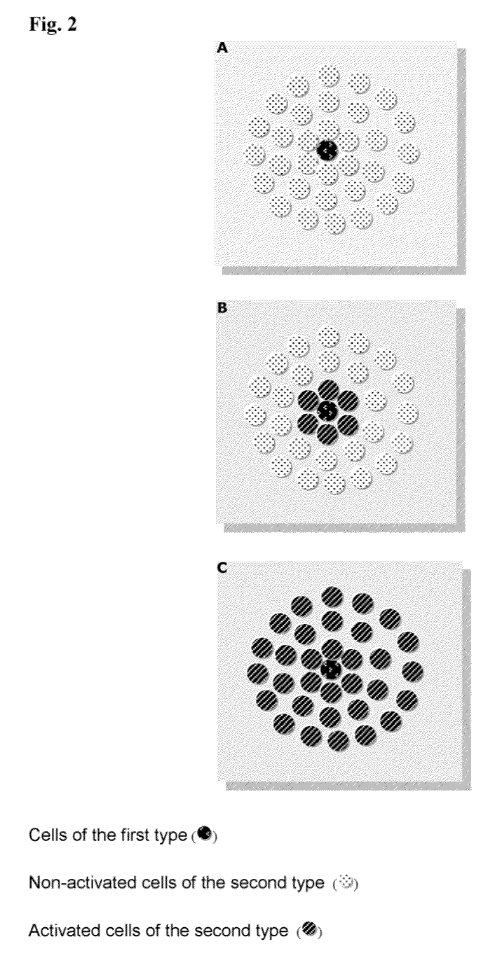

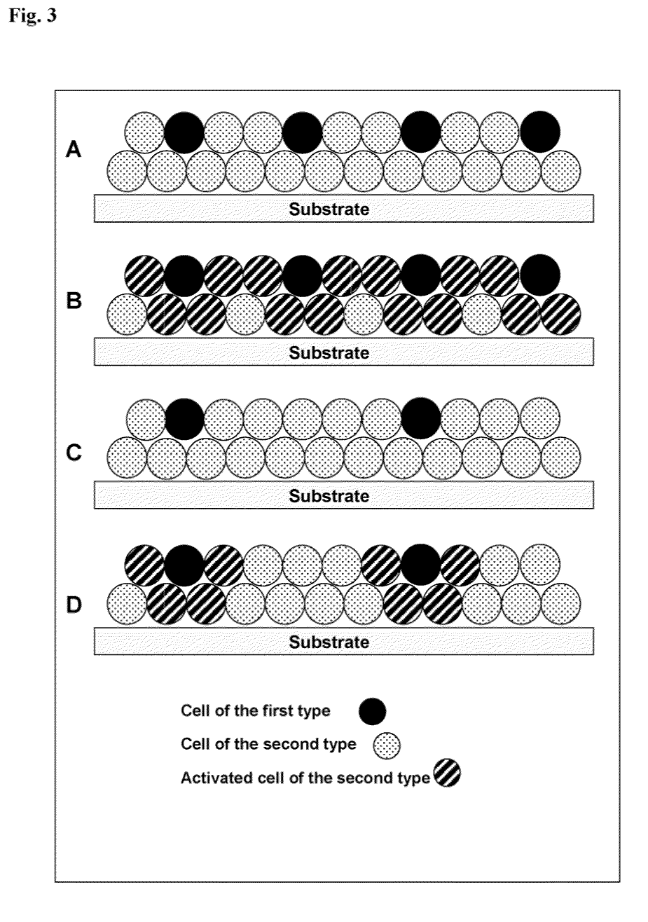

Moreover, the use of a nano plotter enables advantageously to apply the cells of the first, the second and optionally the third type in a spatial arrangement relative to one another on the shape-stable substrate which enhances the amplifying effect of the method additionally (see FIG. 2). In this way, by selection of the quantity ratio of the cells of the first type to cells of the second type as well as optionally cells of the third type as well as the selection of the arrangement of the cells relative to one another the amplification can be affected in a targeted fashion.

Some arrangements of the immobilized yeasts are schematically illustrated in FIGS. 2 and 3.

According to the embodiment of claim 33 in the cells of the first and/or second and/or third type the Afr1 p protein (alpha-factor receptor regulator 1) is inactivated.

As a response to the pheromone induction, a complex mating program (“mating response pathway”) is activated in the cells (Leberer et al, (1997). Pheromone signaling and polarized morphogenesis in yeast. Current Opinion in Genetics & Development 7:59-66.). Mating-specific genes are induced and the cell cycle is arrested. Subsequently, a targeted growth (mating projection) of the cells-to the source of the pheromone, for example, the mating partner, takes place (Jackson et al. (1991). S. cerevisiae α pheromone receptors activate a novel signal transduction pathway for mating partner discrimination. Cell 67: 389-402.; Jackson et al. (1993) Polarization of yeast cells in spatial gradients of α-mating factor. Proc. Natl. Acad. Sei. USA 90: 8332-8336).

This “extension” of the cells is also referred to as “shmoo” (Mackay and Manney, (1974). Mutations affecting sexual conjugation and related processes in Saccharomyces cerevisiae. I. Isolation and phenotypic characterization of nonmating mutants. Genetics 76: 255-271.).

Afr1p (“alpha-factor receptor regulator”) is responsible for the formation of “shmoo” projections during mating of Saccharomyces cerevisiae (Konopka, (1993). AFRI acts in conjunction with the alpha-factor receptor to promote morphogenesis and adaptation. Mol Cell Biol. 13: 6876-6888.). Δafr1 mutants can no longer form normal mating projections. Otherwise, Δafr1 mutants exhibit however a normal sensitivity relative to a stimulation with a-factor (Konopa, 1993). Accordingly, the deletion of the AFR1 gene can be utilized in order to prevent escape from the embedding matrix of the yeast cells by means of “shmoo” projections without the pheromone signal pathway being impaired because cells in which this protein is inactivated, can still receive pheromone signals but do not form mating projections (shmoo) and upon detection of pheromone can no longer mate with the cells of the other mating type. This prevents advantageously that the cells of the method according to the invention in response to a specific primary signal and the thus effected secretion of pheromone cannot grow out and damage a matrix in which they are embedded. Moreover, they cannot mate with one another and become unsuitable for the method.

For the deletion of the AFR1 reading frame preferably a HIS5⁺ deletion cassette is utilized that replaces by double homologous recombination the AFR1 reading frame in the genome. The HIS5 cassette is provided for this purpose at the 5′ terminus and the 3′ terminus by means of SFH-PCR (SFH “short flanking homology region”) with flanking sequences of 40 by length of the AFR1 gene in accordance with Wach et al. (Wach et al. (1997) Heterologous HIS3 marker and GFP reporter modules for P CR-tar geling in Saccharomyces cerevisiae. Yeast 13: 1065-1075).

The α-factor is cleaved by the specific protease Bar1p of Saccharomyces cerevisiae and thus inactivated. Bar1p is secreted and required for a correct mating of the yeast cells. MATa cells in which Bar1p is inactivated show a significantly increased sensitivity relative to the α-factor. In order to increase the sensitivity and response time of the amplification system, according to the embodiment of claim 34 cells are used in which the Bar1p protein is inactivated or the corresponding gene has been deleted (Ballensiefen W and Schmitt H. D. (1997) Periplasmic Bad protease of Saccharomyces cerevisiae is active before reaching its extracellular destination. Eur J Biochem 247(1):142-7; Chan R. K. and Otte C. A. (1982) Physiological characterization of Saccharomyces cerevisiae mutants supersensitive to G1 arrest by a factor and alpha factor pheromones. Mol Cell Biol 2(I):21-9; Barkai N, et al. (1998) Protease helps yeast find mating partners. Nature 396(6710):422-3; Sprague G. F. Jr and Herskowitz I. (1981) Control of yeast cell type by the mating type locus. I. Identification and control of expression of the α-specific gene BAR1. J Mol Biol 153(2):305-21).

According to the embodiment of claim 35, as yeast cells those cells are used that are genetically modified such that their growth can be controlled in a targeted fashion. This enables advantageously that for the manufacture of the device according to the invention the required quantity of yeast cells can be cultured under so-called permissive conditions and after embedding of the yeast cells into a matrix the yeast cells, by adjusting the restrictive conditions, are prevented from further division. In this way, the pressure caused by the vegetative growth of the cells within the matrix is advantageously avoided that would impair the durability of the devices as well as would exert stress onto the immobilized cells and negatively affect their vitality.

Yeast cells are preferably suitable in which the activity of a gene that acts on the cell cycle can be controlled in a targeted fashion. Especially preferred are yeast cells in which the activity of the CDC28 gene is controlled in a targeted fashion. The CDC28 gene is required by the yeast cell in order to be able to divide. When the gene is not present, the cell may survive but cannot divide.

The control of the gene activity is realized for example by the so-called tet on system. In this connection, a yeast cell, in which the endogenous CDC28 gene is deleted (a so-called Δcdc28 cell), is transformed by a DNA construct that contains the coding sequence of the CDC28 gene under the control of a tet-responsive promoter. At the same time, the construct contains the coding sequence of the reverse tetracycline-controlled transactivator (rtTA) under the control of a constitutive promoter.

Such genetically modified yeast cells express continuously the reverse tetracycline-controlled transactivator. The latter can bind only in the presence of a tetracycline antibiotic such as, for example, doxycycline, to the tet-responsive promoter and suppress the expression of the gene that is under the control of the tet-responsive promoter. In order to grow the cells, the culturing medium has added to it a tetracycline antibiotic and thus provides permissive conditions. During or after embedding of the cells into the xerogel the tetracydine antibiotic is washed out and in this way restrictive conditions for the yeast are provided. The reverse tetracycline-controlled transactivator can no longer activate the expression of the CDC28 gene. The yeast cells can therefore no longer divide.

According to the embodiment of claim 36, as yeast cells cell-cycle yeast mutants (cdc; cell division cycle) are used that upon permissive temperature grow normally and at restrictive temperature stop their growth. Several temperature-sensitive (ts) alleles of the CDC28 gene of Saccharomyces cerevisiae are known. For example, six different ts alleles have been identified that allow for a normal growth of the yeast at 23° C. but prevent growth at 37° C. (LOrincz and Reed, 1986). Moreover, there are temperature sensitive mutations known in which the permissive temperature is higher than the restrictive temperature. These are referred to as cold sensitive, cs, mutations. Advantageously, by using such mutants at permissive temperature, first the required biomass can be generated while the yeast cells stop growth at restrictive temperature. When such mutants are used for the device according to the invention, for thermo-sensitive mutants the cells can advantageously be grown up to the point of reaching the desired biomass at approximately 25° C. and then embedded. At restrictive temperature of e.g. 37° C.—a temperature that is ideal for fermentation of Escherichia coli—no growth of the yeasts takes place even though the cells are physiologically active (Lörincz, A and Reed, S.I. Sequence analysis of temperature-sensitive mutations in the Saccharomyces cerevisiae gene CDC28. Mol. Cell. Biol. (1986) 6:4099-4103). Yeasts that contain the temperature-sensitive alleles cdc28-4, cdc28-6, cdc28-9, cdc28-13, cdc28-16, cdc28-17, cdc28-18 and cdc28-19 are preferably used.

For applications in which the yeasts are to be used at low temperatures, for example, room temperature, cold-sensitive mutants are used that are grown at high temperatures and after embedding are kept at low temperatures and therefore have no division activity anymore:

According to claim 37, the cells are coupled with at least one source for electromagnetic radiation and at least one photodetector so that electromagnetic radiation impinges on the yeast cells and the fluorescence is measured by the photodetector.

According to the embodiment of claim 38, the photodetector is a solid-state image sensor with photoresistors, photo diodes or photo transistors and the solid-state image sensor is connected to a data processing system.

A solid-state image sensor is a flat and matrix-shaped arrangement of opto-electronic semiconductor elements as photoelectric receivers. The color of the cells and its intensity can be converted into equivalent electrical signals so that processing in the data processing system can take place.

The cells according to the embodiment of claim 39 are disposed on at least one surface in a transparent measuring cells. The latter comprises moreover devices for supplying and removing the medium.

According to the embodiment of claim 40, the measuring cell is coupled to a heating device.

A source for electromagnetic radiation and a photodetector are arranged according to the embodiment of claim 41 such that electromagnetic radiation emitted by the particles are imaged on the photo receiver.

According to the embodiment of claim 42, the signal detector is a photodetector. The photodetector is a solid-state image sensor with photoresistors, photo diodes or photo transistors wherein it is connected to a data processing system. The solid-state image sensor is a flat and matrix-shaped arrangement of opto-electronic semiconductor elements as photoelectric receivers. The color and its intensity of the yeast cell can be converted into equivalent electrical signals so that processing in the data processing system can take place.

According to the embodiment of claim 43, in the beam path downstream of the electromagnetic radiation and/or upstream of the photodetector at least one beam-shaping or at least one beam-influencing optical device or at least a combination thereof are provided. In this way, the light beams of the yeast cells can be focused onto the photodetector so that a safe evaluation even of weak changes of the light is enabled.

The yeast cells according to the embodiment of claim 44 are coupled to an optical radiation source such that the radiation reaches the yeast cells and the cells fluoresce. The radiation source provides preferably electromagnetic radiation in the form of light in the visible range and the adjoining wavelength range in the infrared and ultraviolet ranges. Preferably, this is an electromagnetic radiation source that emits light at a defined wavelength. The wavelength of the radiation source depends on the excitation spectrum of the fluorescent proteins.

An aspect of the invention is moreover also a method according to claim 48 for detection and amplification of a primary signal by utilizing cells, i.e., a method, wherein
a) in cells of a first type a gene that is responsible for the synthesis of the signal molecule is under the control of a promoter that is regulated by the primary signal,
b) in cells of a second type a specific gene is under the control of a promoter that is regulated by the secreted signal molecule,

so that
by a primary signal received by a cell of the first type the secretion of the signal molecule is induced,
the primary signal is amplified by the signal molecule-controlled expression of the specific gene by the cells of the second type.

According to the embodiment of claim 49, additionally
c) cells of a third type are used in which a gene that is responsible for the synthesis of the signal molecule is under the control of a promoter that is regulated by a signal molecule that is secreted by the cells of the first type,

so that
by a primary signal received by a cell of the first type the secretion of the signal molecule in the first cell is induced,
by the secretion of the signal molecule by the cells of the first type in the cells of the third type the secretion of the signal molecule is induced, and
the primary signal is amplified by the signal molecule-controlled expression of the specific gene by the cells of the second type.

Special embodiments of the components utilized in the method according to the invention are carried out in the same sense as the special embodiments of the features of the device according to the invention according to the claims 3 to 44. According to the embodiment of claim 50, therefore the method is performed by using at least one device with at least one feature of one of the claims 3 to 44.

Based on the following figures and embodiments the invention will be explained in more detail.

In this connection it is shown in:

FIG. 1 Schematic illustration of gene-technologically modified Saccharomyces cerevisiae yeast cells of the mating type a and of the mating type a according to Example 1.

FIG. 2 Device for signal amplification by means of the pheromone system of yeast.

The immobilization of defined cell quantities is realized by means of a nano plotter.

Cells of the first type that produce in response to a certain primary signal (for example, limitations of nutrients in the medium) the yeast pheromone α-factor, are surrounded concentrically on a surface (for example, a glass object holder) by cells of the second type. In the cells of the second type a marker gene, for example, coding for GFP (green-fluorescent protein) is under the control of a pheromone-induced promoter, preferably the FIG1 promoter (A-C). Upon induction of the yeast pheromone expression of the marker gene takes place and thus fluorescence of the cells of the second type (B-C). The expression of the pheromone depends on the level of limitation. Accordingly, for minimal limitation the cells of the second type in immediate environment of the cells of the first type (B) show fluorescence, upon strong limitation also cells that are father removed may even show fluorescence (C).

FIG. 3 Schematic layer configuration with different densities of sensor cells

Cells of the second type that in response to α-factor produced by the cell of the first type generate a readable signal (“activated responsive cell”) are immobilized on a sensor surface (A, C). For a higher proportion of cells of the first type the secretion of pheromone produces a stronger signal (B) than for a reduced proportion of cells of the first type (D). Such layer systems can also be combined in a pyramid configuration.

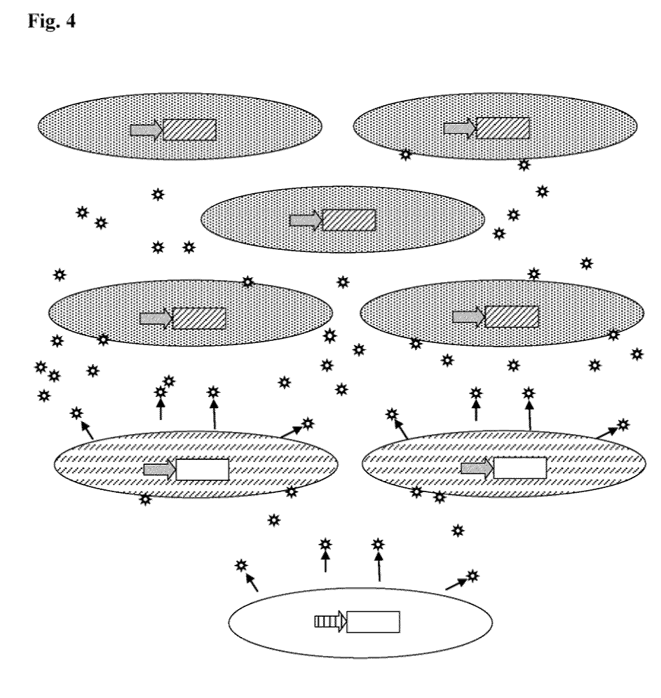

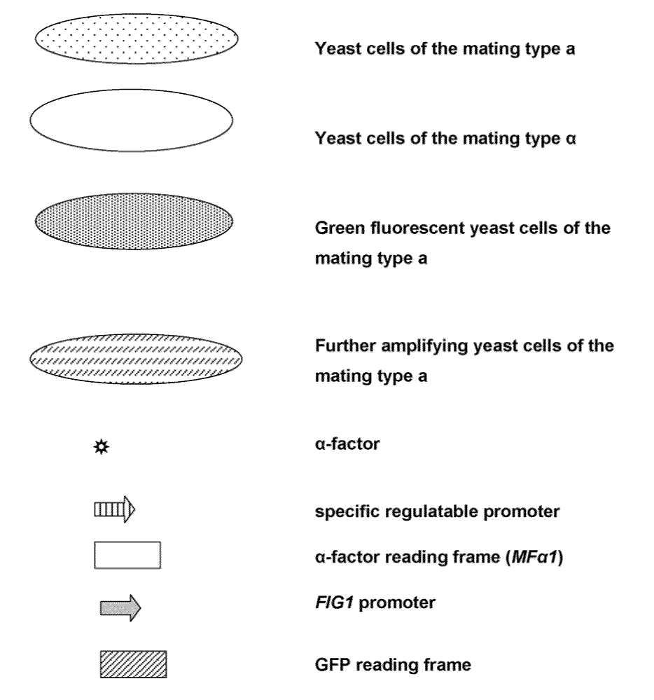

FIG. 4 Schematic illustration of an additional signal amplification according to Example 3.

### EXAMPLE 1

Signal Amplification by Utilization of the Pheromone System of Yeast.

Saccharomyces cerevisiae yeast cells of the mating type a recognize as cells of the first type by means of a receptor an incoming primary signal. Receptors induce directly or by means of intermediately positioned signal cascades the transcription of the promoter. The MFα1 reading frame coding for the α-factor is cloned under the control of the promoter so that the yeast cell of the mating type α, as a response to an incoming primary signal, secretes the pheromone α-factor into the environment. The production of such a yeast cell of the first type is disclosed in the following in an exemplary fashion for use in monitoring bioavailable phosphorus. In this connection, the yeast cells of the first type (sensor cells) react sensitively to a limitation of phosphorus. The gene YAR071W is transcribed specifically much more strongly in case of phosphorus limitation (Boer et al., (2003). The genome-wide transcriptional responses of Saccharomyces cerevisiae grown on glucose in aerobic chemostat cultures limited for carbon, nitrogen, phosphorus, or sulfur. J. Biol. Chem. 278:3265-3274.) The region of the up-regulating gene YAR071W comprising 1,000 base pairs and positioned upstream is amplified by means of the specific primer SEQ NO. 5 and SEQ NO. 6 of Table 2 by PCR of genomic DNA of Saccharomyces cerevisiae. By means of the primers, the sequence is expanded by a 5′-terminal recognition sequence for Sad and at the 3′-terminus by a recognition sequence for Spel. By means of these recognition sequences a directed incorporation into the “high copy number” vector p426, referred to in the following as p426YAR071W, is accomplished. In the second step, the reading frame of the MFα1 gene is cloned into the plasmid p426YAR071W. For this purpose, the sequence of the MFα1 reading frame is amplified by the primers SEQ NO. 7 and SEQ NO. 8 (see Table 2) from genomic DNA of Saccharomyces cerevisiae that expand the fragment at the 5′ terminus by one Spel restriction site and at the 3′-terminus by one Sa/1 restriction site. Subsequently, cloning of the fragment with the aforementioned restriction sites into the vector p426YAR071W, referred to in the following as p426YAR071W-MFalpha1, is carried out. The correct sequence of the cloned fragments is checked and validated by means of DNA sequence analysis. The vector p426 contains a URA3 marker of Saccharomyces cerevisiae for selection in ura-auxotrophic strains. The resulting construct p426YAR071W-MFalpha1 is transformed, for example, into the yeast strain BY4742 (MATα, his3Δ1, leu2Δ0, lys2Δ0, ura3Δ0) and positive transformants are selected. For phosphorus limitation, specifically the expression of the α-factor is induced in sensor cells that are provided with the plasmid p426YAR071W-MFalpha1.

TABLE 2
Primer for the production of the sensor plasmid
p426YAR071W-MFalpha1. Segments that are
homologous to genomic target sequences are marked
in bold, recognition sequences
for restriction endonucleases are underlined.

| SEQ | | |
|---|---|---|
| NO. | Name | Sequence (5′ → 3′) |
| 5 | YAR071W-for-SacI | TATTATGAGCTCGGTGCTGTGACCGTTTCC |
| | | AATACG |
| 6 | YAR071W-rev-SpeI | TATTATACTAGTTGGTATTTCTGATGATGTT |
| | | CTTGCTCTCTTTG |
| 7 | MFalpha1-for- | TATTATACTAGTATGAGATTTCCTTCAATTT |
| | SpeI | TTACTGCAG |
| 8 | MFalpha1-rev- | TATTATGTCGACTTAGTACATTGGTTGGCC |
| | SalI | GGG |

The genes MFα1 and MFα2 that authentically code for the α-factor are deleted in the same strain. In this way, it is ensured that the a factor is exclusively formed and secreted when the primary signal to be detected is present.

For the deletion of the reading frame of MFα1 and MFα2, for example, in the α-yeast strain BY4742 (MATα, his3Δ1, leu2Δ 0, lys2d0, ura3Δ0) the marker cassettes natMX6 and hphMX6 are used that impart resistance against the antibiotics nourseothricin or hygromycin B. The natMX6 cassette is amplified by SFH-PCR by means of the primer SEQ NO. 9 and SEQ NO. 10 of Table 3. The 5′ end areas of the primers (50 bases each) are homologous to the flanking sequences of the MFα1 reading frame in the genome of Saccharomyces cerevisiae. The 3′ end areas of the primers (20 bp) are homologous to the ends of the natMX6 cassette. As DNA templates for the SFH-PCR the plasmid pFA6a-natMX6 is provided. Subsequently, the yeast cells are transformed with the SFH fragment. Transformants in which the fragment is stably integrated by means of double-homologous recombination into the genome are selected from medium containing nourseothricin and the correct integration of the deletion cassette is confirmed by means of diagnostic PCR. Subsequently, the deletion of the reading frame of MFα2 in the generated Δmfαl yeast strain is carried out. For this purpose, in analogy to the first deletion, an SHF fragment is amplified with the primers SEQ NO. 11 and SEQ NO. 12 (see Table 3) and the hphMX6 cassette (DNA template pFA6a-hphMX6) and transformed into Δmfα1 yeast cells. The 5′ end areas of the primers are homologous to the flanking sequences of the MFα2 reading frame in the genome of Saccharomyces cerevisiae. The selection of positive transformants is realized on medium containing hygromycin B and the correct integration of the hygromycin-resistance cassette in the Δmfα1-Δmfα2 yeast strain is checked by diagnostic PCR.

TABLE 3
Primer for the deletion of the reading frame of
MFα1 and MFα2 of Saccharomyces cerevisiae. The
unmarked primer sequence characterizes areas that
are homologous to the genomic DNA of Saccharomyces
cerevisiae. Segments that are homologous to the
deletion cassette are marked in bold.

| SEQ | | |
|---|---|---|
| NO. | Name | Sequence (5′ → 3′) |
| 9 | MFalp1_F2 | AAGAAGATTACAAACTATCAATTTCATACACAATA |
| | | TAAACGATTAAAAGACGGATCCCCGGGTTAATTAA |
| 10 | MFalp1_R1 | TGGGAACAAAGTCGACTTTGTTACATCTACACTGT |
| | | TGTTATCAGTCGGGCGAATTCGAGCTCGTTTAAAC |
| 11 | MFalp2_F2 | TTACTACCATCACCTGCATCAAATTCCAGTAAATT |
| | | CACATATTGGAGAAACGGATCCCCGGGTTAATTAA |
| 12 | MFalp2_R1 | ATGAACGTGAAAGAAATCGAGAGGGTTTAGAAGTA |
| | | GTTTAGGGTCATTTTGAATTCGAGCTCGTTTAAAC |

The Saccharomyces cerevisiae yeast cells of the mating type a present in the same batch as cells of the second type are modified in that they contain the reading frame coding for EGFP under the control of the FIG1 promoter.

For this purpose, 1,000 by at the 5′ terminus of the open reading frame of FIG1 by using the primers FIG. 1-for (SEQ NO. 1) and FIG. 1-rev (SEQ No. 2) (see Table 1) are

PCR-amplified, purified, cut with the restriction endonucleases Sad and Spel and cloned into the S. cerevisiae vector p426. The thus produced vector (p426FIG1) was cut with the enzymes Sa/1 and EcoR1.

The reading frame coding for EGFP was PCR-amplified by means of the primers EGFPEcofor (SEQ NO. 13) and EGFPSalrev (SEQ NO. 14) and the fragment of 744 by was cut with the enzymes Sail and EcoR1, purified, and used for ligation into the vector p426FIG1.

TABLE 4
Primer for the amplification of the open reading
frame of EGFP. The letters in bold print delimit
the coding reading frame of the EGFP gene. In
italics the recognition sequences of the restric-
tion endonucleases EcoRI and SalI are indicated
that are used for cloning into the vector p426FIG1.
The first six bases serve to protect the primer.

| SEQ | | |
|---|---|---|
| NO. | Primer | Sequence (5′ → 3′) |
| 13 | EGFP | TAT TAT GAA TTC ATG GTG AGC AAG GGC |
| | Ecofor | GAG GAG |
| 14 | EGFPSalrev | TAT TAT GTC GAC TTA CTT GTA CAG CTC |
| | | GTC CAT GCC G |

The DNA sequence of the cloned reading frame was verified by DNA sequence analysis. Accordingly, the vector p426FIG1-EGFP was available for the transformation of yeast cells. The transformation of the completed vector in yeast cells of the mating type a was realized as described above for the yeast cells of the mating type a.

The genes (MFA1 and MFA2) coding for the a-factor are deleted in order to prevent secondary effects on the a-cells. The deletion was carried out in analogy to the method disclosed in connection with the genes MFα1 and MFα2.

When after induction of the specifically regulated promoter the then generated and secreted α-factor reaches the surrounding Saccharomyces cerevisiae yeast cells of the mating type a, in these cells the transcription of the reading frame coding for GFP is greatly induced by means of the FIG1 promoter. This results in a green fluorescence of the yeast cells that can be read out by sensors. The intensity of the green fluorescence can be increased proportionally to the number of the a-cells surrounding the α-cells.

### Example 2

Additional Signal A

The genetic modification of Saccharomyces cerevisiae yeast cells of the mating type a as cells of the first type is realized as in Example 1.

As cells of the second type Saccharomyces cerevisiae yeast cells of the mating type a are modified as in Example 1.

As cells of the third type, cells of the mating type a have been modified such that they act as a further amplifier. For this purpose, a reading frame which codes for the pheromone a-factor is under the control of the FIG1 promoter.

For this purpose, the FIG1 promoter is PCR-amplified and cloned into yeast vector p426 as disclosed in Example 1. Subsequently, the gene MFα1 is inserted into the same vector at the 3′ end of the FIG1 promoter.

When the α-factor secreted primarily by the action of a signal molecule reaches the surrounding a-cells (cells of the third type), by induction of the FIG1 promoter the formation of further a-factor molecules is caused in these cells, i.e., further amplification (FIG. 4). The level of amplification can be determined by the selection of the ratio of cells of the mating types a and a (FIG. 1).

What is claimed is:

1.-50. (canceled)

51. A device for detection and amplification of a primary signal, comprising:
a) cells of a first type in which a gene that is responsible for the synthesis of a signal molecule is under the control of a promoter that is regulated by the primary signal;
b) cells of a second type in which a specific gene is under the control of a promoter that is regulated by the secreted signal molecule;
wherein by a primary signal received by a cell of the first type the secretion of the signal molecule is induced; and
wherein the primary signal is amplified by the signal molecule-controlled expression of the specific gene by the cells of the second type.

52. The device according to claim 51, further comprising cells of a third type in which a gene that is responsible for the synthesis of the signal molecule is under the control of a promoter that is regulated by the signal molecule that is secreted by the cells of the first type,
wherein by a primary signal received by a cell of the first type the secretion of the signal molecule is induced in the first cell,
wherein by the secretion of the signal molecules by the cells of the first type in the cells of the third type the secretion of the signal molecule is induced and a pre-amplification of the primary signal is effected, and
wherein the primary signal is amplified by the signal molecule-controlled expression of the specific gene by the cells of the second type.

53. The device according to claim 51, wherein the cells of the first type, the cells of the second type, or the cells of the first and second types are yeast cells.

54. The device according to claim 52, wherein the cells of the third type are yeast cells.

55. The device according to claim 51, wherein the cell of the first type are modified gene-technologically so that the cell of the first type expresses a receptor system specific for the primary signal.

56. The device according to claim 51, wherein the cell of the second type is modified gene-technologically so that the cell of the second type expresses a receptor system specific for the signal molecule.

57. The device according to claim 51, wherein the signal molecule is a pheromone.

58. The device according to claim 57, wherein the gene coding for the pheromone is the MFα1 gene, the MFα2 gene, the MFA1 gene or MFA2 gene of Saccharomyces cerevisiae.

59. The device according to claim 51, wherein the specific gene is responsible for the formation of a signal molecule that is different from the signal molecule secreted by the cells of the first type wherein the specific gene codes for a marker protein or a fluorescent protein.

60. The device according to claim 51, wherein the promoter that can be regulated by a primary signal is a promoter that is regulated nitrogen-, phosphate- or sulfur-specific.

61. The device according to claim 51, wherein the cells are disposed in a porous organic or inorganic gel.

62. The device according to claim 61, wherein the inorganic gel is a silicon dioxide xerogel and the xerogel with the cells disposed therein is disposed on a substrate with increased mechanical stability.

63. The device according to claim 51, wherein the cells are coupled with at least one source of electromagnetic radiation and at least one photodetector such that electromagnetic radiation impinges on the cells and the fluorescence is measured by the photodetector.

64. A method for detection and amplification of a primary signal, the method comprising the steps of:
in cells of a first type, subjecting a gene that is responsible for the synthesis of a signal molecule to the control of a promoter that is regulated by the primary signal;
in cells of a second type, subjecting a specific gene to the control of a promoter that is regulated by the secreted signal molecule;
inducing by a primary signal received by a cell of the first type the secretion of the signal molecule; and
amplifying and/or converting the primary signal the primary signal by the signal molecule-controlled expression of the specific gene by the cells of the second type.

65. The method according to claim 64, further comprising:
providing cells of a third type in which a gene that is responsible for the synthesis of the signal molecule is under the control of a promoter that is regulated by the signal molecule that is secreted by the cells of the first type,
inducing by a primary signal received by a cell of the first type the secretion of the signal molecule in the first cell;
inducing by the secretion of the signal molecules by the cells of the first type in the cells of the third type the secretion of the signal molecule;
amplifying the primary signal by the signal molecule-controlled expression of the specific gene by the cells of the second type.

66. The method according to claim 64, wherein the primary signal is generated by a limitation of nutrients in a culture medium and therefore the limitation of the nutrients is detected.

67. The method according to claim 64, wherein the primary signal is generated by a loading of cells with stress-inducing agents and therefore the loading of the cells is detected.

68. The method according to claim 64, wherein the primary signal is generated by substances in aqueous solution and therefore the substances are detected.
